(19) United States
(12) Patent Application Publication
(10) Pub. No.: US 2012/0012539 A1
(43) Pub. Date: Jan. 19, 2012

(54) WATER TREATMENT APPARATUS AND METHOD FOR USING SAME

(75) Inventor: WILLIAM GREEN, MARTINSBURG, WV (US)

(73) Assignee: QIAGEN GAITHERSBURG INC., Gaithersburg, MD (US)

(21) Appl. No.: 12/986,816

(22) Filed: Jan. 7, 2011

Related U.S. Application Data
(60) Provisional application No. 61/293,283, filed on Jan. 8, 2010.

(51) Int. Cl. C02F 1/00; C02F 1/42; C02F 1/28; C02F 1/30; C02F 9/02; C12M 1/34; C02F 1/44; B01D 29/07; C02F 101/20; C02F 101/30; C02F 101/10; C02F 101/12; C02F 101/16

(52) U.S. Cl. 210/767; 210/323.1; 210/196; 435/287.1

(57) ABSTRACT

A water treatment apparatus with a first filtration system and a second filtration system is disclosed. The first filtration system provides purified water to an automated assay device. The second filtration system receives wastewater from an outlet and removes contaminants from the wastewater which allows for direct disposal of the wastewater via environmentally responsible means. An automated assay device comprising the water treatment apparatus and a method of treating water used by the automated assay device is also disclosed.

### REFERENCE TO RELATED APPLICATIONS

This application claims priority to U.S. Provisional patent application No. 61/293,283, filed on Jan. 8, 2010, which is incorporated herein by reference in its entirety.

### FIELD

A water treatment apparatus removably attached to an automated assay device is disclosed. More specifically, a water treatment apparatus removably attached to an automated assay device that provides purified water to and wastewater removal from the automated assay device is disclosed.

### BACKGROUND

Automated assay devices or analyzers often require the use of purified water and the removal of wastewater. Existing automated assay devices require the steps of adding de-ionized water containers and removing wastewater containers. These steps of adding de-ionized water and removing wastewater containers lengthen the cycle time of such automated assay devices. In addition such containers are costly and require laboratory storage space.

Therefore, there exists a need for a water treatment apparatus which improves the cycle time of existing automated assay devices by eliminating the steps of adding de-ionized water containers and removing wastewater containers. Additionally, there exists a need for a water treatment apparatus which reduces cost to clinical laboratories by eliminating the need to purchase and store de-ionized water and wastewater removal containers.

### SUMMARY

One aspect relates to a water treatment apparatus removably attached to an automated assay device using a direct feed of purified water and an outlet for wastewater. The apparatus has a first filtration system and a second filtration system. The first filtration system provides purified water to the direct feed of the assay device. The second filtration system receives wastewater from the outlet and removes contaminants from the wastewater allowing for direct drain disposal of the wastewater via environmentally responsible means.

Another aspect relates to a method of treating water used by an automated assay device using a direct feed of purified water and an outlet for wastewater. The method comprises (a) moving impure water into a first filtration system removably attached to the automated assay device, wherein the first filtration system provides purified water; (b) moving the purified water produced by the first filtration system to the direct feed of the automated assay device; and (c) moving wastewater from the outlet on the automated assay machine to a second filtration system removably attached to the automated assay device, wherein the second filtration system removes contaminants from the wastewater allowing for direct drain disposal via environmentally responsible means.

### DETAILED DESCRIPTION

An apparatus and method of treating water used by an automated assay device is disclosed, wherein purified water for an automated assay device is generated by a first filtration system and wastewater generated by the first filtration system is second filtration system.

In one aspect, the first filtration system is adapted to receive impure water from a water supply, to convert the impure water into purified water, and to supply the purified water to the apparatus.

As used herein, the term “impure water” shall refer to any water that contains at least one contaminant in concentrations sufficient to interfere with the particular laboratory or clinical application performed by the automated assay device. By way of example and not limitation, the “impure water” may be tap water, grey water, well water, de-ionized water (diH₂O), distilled water (dH₂O), and/or double distilled water (ddH₂O).

As used herein, the term “contaminant” shall refer to any substance that interferes with the particular laboratory or clinical application performed by the automated assay device. By way of example and not limitation, the “contaminant” may be selected from the group consisting of DNAse; RNAse; endotoxin; particulate matter; microorganisms; viruses; ions, including but not limited to calcium (Ca²⁺), chloride (Cl⁻), magnesium (Mg²⁺), bicarbonate (HCO₃⁻), sodium (Na⁺), nitrate (NO₃⁻), potassium (K⁺), carbonate (CO₃²⁻), iron (Fe²⁺), and sulfate (SO₄²⁻); organic compounds; nucleic acids; cellular waste and/or debris; body materials and/or fluids; and/or protein. Notwithstanding this, the precise contaminants of interest will depend on the assay being performed and will be apparent to the person having ordinary skill in the art.

As used herein, the term “purified water” shall refer to any water that has been treated such that the concentration of contaminants is reduced to a point at which the water is suitable for the particular laboratory or clinical application performed by the automated assay device. A number of organizations have established standards for characterizing the purity of water, including but not limited to the following standards: International Organization for Standardization specification for water for laboratory use ISO 3696 Grade I, Grade II, and Grade III; American Society for Testing and Materials (ASTM) D1193-91 Type I, Type II, Type III, Type IV, Type A, Type B, and Type C; National Committee for Clinical Laboratory Standards (NCCLS) Type I, Type II, and Type III; European Pharmacopoeia (EP); and US Pharmacopoeia (USP). These standards are useful for describing the requirements for purified water and are fully incorporated herein by reference. Additionally, purified water can be characterized as being RNAse-free, DNAse-free, endotoxin-free, de-ionized (diH₂O), distilled (dH₂O), double distilled (ddH₂O), and/or reverse osmosis (RO—H₂O). However, the precise requirements for purified water will always depend on the application to be performed by the automated assay device, irrespective of whether the water does or does not satisfy any or all of these standards.

In one embodiment, the first filtration system generates purified water by subjecting impure water to at least one process selected from the group consisting of: filtration, including but not limited to particulate filtration, sediment filtration, microfiltration, ultrafiltration, and nanofiltration; reverse osmosis (RO); ion exchange, including but not limited to two-bed deionization, mixed-bed deionization, organic scavenging, and base-exchange softening; carbon adsorption; and irradiation, such as by ultraviolet (UV) light.

In another embodiment, the first filtration system comprises a filter apparatus and a deionization apparatus, wherein impure water passes first through the filter apparatus, then through the deionization apparatus.

In one embodiment, the filter apparatus is selected to remove insoluble particulates and larger soluble molecules that would interfere with reverse osmosis from the water. In one embodiment, the filter apparatus comprises a pre-filter. The pre-filter comprises at least one particulate filter and may further comprise a carbon adsorption apparatus for removing organic molecules. In another embodiment, the filter apparatus comprises at least one size-exclusion filter. In yet another embodiment, the pre-filter may comprise multiple microporous membranes with gradually decreasing pore sizes, arranged such that the filter membrane having the largest pore size is the first to contact the impure water and the membrane having the smallest average pore size is the last to contact the impure water. In one embodiment, the size exclusion filter is suitable for performing microfiltration, ultrafiltration, and/or nanofiltration. The filter apparatus may further comprise a carbon adsorption apparatus, which may be disposed at any point in the filter apparatus. In one embodiment, the carbon adsorption apparatus is disposed in the pre-filter. In another embodiment, the carbon adsorption apparatus is disposed between the pre-filter and the size-exclusion filter. In another embodiment, the carbon adsorption apparatus is disposed in the size-exclusion filter. The term “filter apparatus” should not be construed as relating only to a unitary structure housing each component of the filter apparatus. Rather, each component of the filter apparatus may be housed in the same physical structure or in separate structures as desired by the user. In one embodiment, the filter apparatus is placed in fluid communication with the deionization apparatus, such that impure fluid passes through the filter apparatus before passing into the deionization apparatus. The fluid apparatus and deionization apparatus may be comprised within the same physical structure or may be disposed in distinct physical structures.

In another embodiment, the deionization apparatus is selected to remove ions from the impure water that has passed through the pre-filter. In one embodiment, the deionization apparatus comprises a RO apparatus and an ion exchange apparatus. In another embodiment, the deionization apparatus may comprise at least one reservoir for storing water that has passed through the RO apparatus, the ion exchange apparatus, or both. In another embodiment, the deionization apparatus has a three way valve allowing the water exiting the RO apparatus to bypass the deionization apparatus when water of reverse osmosis quality is required.

The first filtration system may further comprise at least one reservoir for storing water that has been treated by the various components of the first filtration system. Such reservoirs may serve multiple functions, including but not limited to: allowing sediment to settle before entering filters; storing water of various qualities such that multiple different qualities of water can be obtained and used by the same instrument; and permitting the selective recirculation of water through a component of the first filtration apparatus. By way of example and not limitation, the first filtration system may comprise: a reservoir disposed before the filter apparatus for allowing large particulate matter to separate from the water before entering the filter apparatus; a reservoir disposed between the filter apparatus and the deionization apparatus for storing water that has passed through the filter apparatus; a reservoir disposed between the RO apparatus and the ion exchange apparatus for storing reverse osmosis quality water; and/or a reservoir disposed after the deionization apparatus for storing deionized water. The reservoirs may be equipped with an auto shut off float valve that will automatically shut off the portion or portions of the first filtration system preceding the reservoir. In this way, high throughput and low throughput filtration systems may be combined without concern of overflow or backup.

The first filtration system may further have monitors that measure the electrical conductivity of water. The conductivity of water correlates to the total dissolved solids (TDS) contained as disassociated ionic species in the water. A greater concentration of TDS results in higher electrical conductivity, while a lower concentration of TDS results in lower electrical conductivity. This association can be used to determine various factors, including but not limited to when the requisite level of water purity has been reached and the relative performance of components of the first filtration device. For example, the monitor may be set to trigger an alarm to indicate when the electrical conductivity is above or below an internally set level, thus indicating that replacement of such components is required.

The second filtration system receives wastewater from an outlet of the automated assay device and removes pollutants from the wastewater allowing for the direct disposal of the wastewater, i.e., disposal of the wastewater without further treatment, via environmentally responsible means.

As used herein, the term “wastewater” shall refer to any aqueous solution generated by the operation of the automated assay device that contains a pollutant.

As used herein, the term “pollutant” shall refer to any compound, molecule, or object that cannot be disposed in a public sewage system. By way of example and not limitation, the pollutant may be: organic compounds; caustic compounds; detergents; and biological wastes, including but not limited to bodily waste and/or fluids, microorganisms, medical waste, cellular waste, nucleic acids, and/or proteins.

By way of example and not limitation, the second filtration system can comprise, consist essentially of, or consist of a plastic cartridge filter used to filter fine particles from the wastewater. By way of example and not limitation, filters may be 10-inch, 20-inch, or 30-inch wound or pleated polypropylene filter cartridges. The filter may alternatively have a FPM seal. In a further embodiment, the filter may be designed to allow compression to seal the filter unit, thereby eliminating the need for a center rod. In yet another embodiment, the filter may comprise a hand removable filter cover having a plug, thereby permitting easy bleeding of the unit. In one embodiment, the filter has a NPT (female) inlet connection to eliminate the chance for broken NPT (male) connections. Exemplary filters which may be used include, but are not limited to, FLT Series Cartridge Filters manufactured by Hayward; and string wound filters provided by Keystone and Omni Filter. The second filtration system may additionally comprise any of the components described for the first filtration system.

In one embodiment, the first filtration system and the second filtration system may be mounted directly on the automated assay device. By way of example and not limitation, a mounting base may be used to mount the second filtration system to an automated assay device; or one or both of the first filtration system and the second filtration system may be integrated into the automated assay device.

In another aspect, a method of treating water used by an automated assay device requiring a direct feed of purified water and an outlet for wastewater is also disclosed. First, the method comprises moving impure water into a first filtration system. The first filtration system provides purified water to the automated assay device. Second, the method comprises moving the purified water produced by the first filtration system to the direct feed of the automated assay device. Third, the method comprises moving wastewater from the outlet on the automated assay machine to a second filtration system attached to the automated assay device. The second filtration system removes contaminants from the wastewater allowing for direct drain disposal via environmentally responsible means.

### EXAMPLES

As one example, a DI-160 DI De-Ionized Water Filtration System (tm associates) is used as a first filtration system and an FLT Series Cartridge Filter (Hayward) is used as a second filtration system in connection with an automated device for determining the presence of HPV in a clinical sample.

The DI-160 DI system purifies impure water, such as tap water, by passing it through a series of three filters, a reverse osmosis membrane, and a de-ionization cartridge. First, the DI-160 DI system is placed in fluid communication to a tap water source. The tap water passes through a 10-inch ¾-inch NPT pre-filter that is a modified carbon block and particulate filter to 50 microns. The pre-filter extends the life of the down line filters and removes gross contaminants from the incoming feed water and has a life of 1500 gallons. The pre-filter is a ¾-inch flow filter and is designed for high pass through flow and to maintain water pressure. Such a pre-filter also reduces 95% of chlorine in the water. After passing through the pre-filter, the water then passes through a 0.5-micron sediment filter. The sediment filter removes excess turbidity or particulate matter that may cause the reverse osmosis membrane to become clogged or plugged up. After passing through the sediment filter, the water passes through a 0.5 carbon block filter for removal of additional organics and chlorine that can damage the reverse osmosis membrane. After passing through the carbon block filter, the incoming feed water passes through a high rejection thin film composite reverse osmosis membrane. Such a membrane removes a substantial percentage (as much as 98%) of most inorganic salts, all micro-organisms and almost all high molecular weight organics in the water. Finally, after passing through the reverse osmosis membrane, the incoming feed water passes through a mixed multi-layered mixed bed de-ionization cartridge. The cartridge removes almost all minerals and organics that may pass through the membrane, producing purified water with >17 Meg Ohm/cm² resistivity. Purified water is then passed into a first reservoir disposed within the automated device, where it can be selectively added to samples being analyzed and removed from the sample as wastewater. The wastewater is stored in a second reservoir disposed within the automated device, which is in fluid communication with a FLT Series Cartridge Filters manufactured by Hayward. The FLT cartridge is mounted on the automated assay device and functions to filter fine particles from the wastewater. The maximum flow rates for both wound and pleated cartridges are 5 GPM per 10-inch of filter cartridge length. The wastewater is then disposed of in a public drain or stored for further processing or use.

The aforementioned filtration systems are retrofitted to a NexGen™ Multiple-Input Analytical System, which is described in U.S. patent application Ser. No. 12/622,131, which is incorporated herein by reference in its entirety. The NexGen™ system is an automated sample processing platform for analyzing biological samples (such as cervical swabs) for the presence of specific nucleic acids, and requires a direct feed of high purity water in order to operate. Moreover, because the NexGen system utilizes clinical samples, the waste products generated thereby are classified as medical waste and may contain, among other this, hazardous chemicals such as tissue fixatives and biological waste, such as viruses. As retrofitted, tap water is fed into the DI-160-DI system for filtration. Purified water generated by the DI-160-DI system is then fed into a direct feed on the NexGen™ system, where it is utilized in an automated assay. Waste water generated by the assay is then direct into an outlet, which feeds into the Hayward FLT cartridge, which decontaminates the waste water and feeds it into an outlet, which may lead into a storage container for further processing, or may lead into a drain for deposit in a public sewage system.

What is claimed is:

1. A water treatment apparatus attached to an automated assay device, wherein the assay device uses a direct feed of purified water and comprises an outlet for wastewater,
wherein the water treatment apparatus comprises a first filtration system and a second filtration system,
wherein the first filtration system is adapted to provide purified water to the direct feed of the assay device, and wherein the second filtration system is adapted to receive wastewater from the outlet and remove a pollutant from the wastewater.

2. The water treatment apparatus of claim 1 wherein:
a. the first filtration system comprises:
(i) an impure water input adapted to be placed in fluid communication with impure water;
(ii) at least a first filter capable of generating purified water from the impure water; and
(iii) a purified water output adapted to place purified water in fluid communication with the direct feed on the automated assay device; and
b. the second filtration system comprises:
(i) a wastewater input adapted to be placed in fluid communication with wastewater generated by the automated assay device;
(ii) at least a second filter capable of removing the pollutant from the wastewater; and
(iii) a disposal outlet adapted to be placed in fluid communication with wastewater that has had the pollutant removed by the second filter.

3. The water treatment apparatus of claim 2 wherein the first filter is capable of removing a contaminant selected from the group consisting of: DNAse; RNAse; endotoxin; particulate matter; a microorganism; a virus; an ion; an organic compound; a nucleic acid; cellular waste and/or debris; a body material and/or fluid; and a protein.

4. The water treatment apparatus of claim 2 wherein the first filter is capable of generating purified water characterized as at least one type of water selected from the group consisting of:
RNAse-free water, DNAse-free water, endotoxin-free water, de-ionized water (diH₂O), distilled water (dH₂O), double distilled water (ddH₂O), and reverse osmosis water (RO—H₂O).

5. The water treatment apparatus of claim 2 wherein the first filter comprises an apparatus capable of performing at least one process selected from the group consisting of:
a. particulate filtration
b. sediment filtration
c. microfiltration,
d. ultrafiltration,
e. nanofiltration;
b. reverse osmosis (RO);
c. ion exchange;
d. carbon adsorption; and
e. irradiation.

6. The water treatment apparatus of claim 5 comprising an apparatus capable of performing an ion exchange process selected from the group consisting of two-bed deionization, mixed-bed deionization, organic scavenging, and base-exchange softening.

7. The water treatment apparatus of claim 5 wherein the apparatus capable of performing an ion exchange process is capable of removing an ion selected from the group consisting of: calcium (Ca²⁺), chloride (Cl⁻), magnesium (Mg²⁺), bicarbonate (HCO₃⁻), sodium (Na⁺), nitrate (NO₃⁻), potassium (K⁺), carbonate (CO₃²⁻), iron (Fe²⁺), and sulfate (SO₄²⁻).

8. The water treatment apparatus of claim 2 wherein the first filter comprises a filter apparatus and a deionization apparatus, wherein impure water passes first through the filter apparatus, then through the deionization apparatus.

9. The water treatment apparatus of claim 8 wherein the filter apparatus is selected to remove insoluble particulates and larger soluble molecules that would interfere with reverse osmosis from the water.

10. The water treatment apparatus of claim 8 wherein the filter apparatus comprises at least one size-exclusion filter.

11. The water treatment apparatus of claim 10 wherein the size exclusion filter is suitable for performing microfiltration, ultrafiltration, and/or nanofiltration.

12. The water treatment apparatus of claim 9 wherein the filter apparatus comprises a pre-filter comprising at least one particulate filter.

13. The water treatment apparatus of claim 12 wherein the prefilter further comprise a carbon adsorption apparatus.

14. The water treatment apparatus of claim 13 wherein the prefilter comprises multiple microporous membranes with gradually decreasing pore sizes, arranged such that the filter membrane having the largest pore size is the first to contact the impure water and the membrane having the smallest average pore size is the last to contact the impure water.

15. The water treatment apparatus of claim 8 wherein the filter apparatus comprises a carbon adsorption apparatus.

16. The water treatment apparatus of claim 15 wherein the carbon adsorption apparatus is disposed in a pre-filter.

17. The water treatment apparatus of claim 15 wherein the carbon adsorption apparatus is disposed between a pre-filter and a size-exclusion filter.

18. The water treatment apparatus of claim 15 wherein the carbon adsorption apparatus is disposed in a size-exclusion filter.

19. The water treatment apparatus of claim 8 wherein the filter apparatus is placed in fluid communication with the deionization apparatus, such that impure fluid passes through the filter apparatus before passing into the deionization apparatus.

20. The water treatment apparatus of claim 8 wherein the deionization apparatus is selected to remove ions from the impure water that has passed through a prefilter.

21. The water treatment apparatus of claim 8 wherein the deionization apparatus comprises a reverse osmosis apparatus and an ion exchange apparatus.

22. In another embodiment, the deionization apparatus may comprise at least one reservoir for storing water that has passed through the reverse osmosis apparatus.

23. The water treatment apparatus of claim 2 wherein the first filtration system further comprises a reservoir.

24. The water treatment apparatus of claim 23 wherein the reservoir is adapted to perform at least one function selected from the group consisting of:
a. allowing sediment to settle before water enters a filter;
b. storing water; and
c. permitting the selective recirculation of water through a component of the first filtration apparatus

25. The water treatment apparatus of claim 24 wherein the reservoir is adapted to store impure water.

26. The water treatment apparatus of claim 24 wherein the reservoir is adapted to store water obtained after impure water passes through at least one apparatus capable of performing at least one process selected from the group consisting of:
a. particulate filtration
b. sediment filtration
c. microfiltration,
d. ultrafiltration,
e. nanofiltration;
b. reverse osmosis;
c. ion exchange;
d. carbon adsorption; and
e. irradiation.

27. The water treatment apparatus of claim 24 wherein the reservoir is adapted to store purified water.

28. The water treatment apparatus of claim 2 wherein the first filtration system further comprises a monitors that measures the electrical conductivity of water passing through the first filtration system.

29. The water treatment apparatus of claim 2 wherein the second filtration system is capable of removing from the wastewater at least one pollutant selected from the group consisting of: an organic compound; a caustic compound; a detergent; and biological waste.

30. The water treatment apparatus of claim 29 wherein the second filtration system comprises a plastic cartridge filter capable of filtering fine particles from the wastewater.

31. The water treatment apparatus of claim 30 wherein the plastic cartridge filter comprises a wound or pleated polypropylene filter cartridge.

32. The water treatment apparatus of claim 31 further comprising at least one an apparatus capable of performing at least one process selected from the group consisting of:
a. particulate filtration
b. sediment filtration
c. microfiltration,
d. ultrafiltration,
e. nanofiltration;
b. reverse osmosis (RO);
c. ion exchange;
d. carbon adsorption; and
e. irradiation.

33. The water treatment apparatus of claim 2 wherein the second filtration system further comprises a reservoir.

34. The water treatment apparatus of claim 33 wherein the reservoir is adapted to perform at least one function selected from the group consisting of:
a. allowing sediment to settle before water enters a filter;
b. storing water; and
c. permitting the selective recirculation of water through a component of the second filtration apparatus

35. The water treatment apparatus of claim 34 wherein the reservoir is adapted to store wastewater.

36. The water treatment apparatus of claim 34 wherein the reservoir is adapted to store water obtained after wastewater passes through at least one apparatus capable of performing at least one process selected from the group consisting of:
a. particulate filtration
b. sediment filtration
c. microfiltration,
d. ultrafiltration,
e. nanofiltration;
b. reverse osmosis;
c. ion exchange;
d. carbon adsorption; and
e. irradiation.

37. The water treatment apparatus of claim 1, wherein the water treatment apparatus is attached to the automated assay device, wherein the first filtration system provides purified water to the direct feed of the assay device, and wherein the second filtration system receives wastewater from the outlet and removes pollutants from the wastewater, allowing for disposal of the wastewater without further treatment.

38. The water treatment apparatus of claim 1, wherein the water treatment apparatus is removably attached to the automated assay device

39. An automated assay device comprising a water treatment apparatus of claim 1.

40. The automated assay device of claim 39 wherein the automated assay device is adapted for high throughput processing of biological samples.

41. The automated assay device of claim 39 wherein the automated assay device generates wastewater comprising an infectious agent and wherein the water treatment apparatus removes the infectious agent from the wastewater.

42. The automated assay device of claim 39 wherein the infectious agent is a virus.

43. A method of treating water used by an automated assay device, wherein the assay device uses a direct feed of purified water and comprises an outlet for wastewater, the method comprising:
a. moving impure water into a first filtration system removably attached to the automated assay device, wherein the first filtration system provides purified water;
b. moving the purified water produced by the first filtration system to the direct feed of the automated assay device; and
c. moving wastewater from the outlet to a second filtration system removably attached to the automated assay device, wherein the second filtration system removes pollutants from the wastewater allowing for disposal of the wastewater without further treatment.
